(19) United States
(12) Patent Application Publication
(10) Pub. No.: US 2022/0142944 A1
(43) Pub. Date: May 12, 2022

(54) TREATMENT OF SKIN DISORDERS WITH TOPICAL TAPINAROF-EGFR INHIBITOR COMPOSITIONS

(71) Applicant: Sol-Gel Technologies Ltd., Ness Ziona (IL)

(72) Inventors: Moshe ARKIN, Kfar Shmaryahu (IL); Marcel ZIGHELBOIM, Kiryat Motzkin (IL); Ori NOV, Tarum (IL); Ofer TOLEDANO, Kfar Saba (IL); Karine NEIMANN, Ness Ziona (IL); Hila HAKAK DJERBI, Tel Aviv (IL)

(73) Assignee: Sol-Gel Technologies Ltd., Ness Ziona (IL)

(21) Appl. No.: 17/582,273

(22) Filed: Jan. 24, 2022

Related U.S. Application Data
(63) Continuation-in-part of application No. PCT/IL2020/050817, filed on Jul. 23, 2020.
(60) Provisional application No. 63/005,353, filed on Apr. 5, 2020.
(60) Provisional application No. 62/877,966, filed on Jul. 24, 2019.

(51) Int. Cl. A61K 31/05; A61K 47/18

(52) U.S. Cl. CPC A61K 31/05; A61K 9/0014; A61K 47/186

(57) ABSTRACT

Disclosed are topical compositions and methods of treatment of a skin or mucosal disorder selected from palmoplantar psoriasis, hereditary palmoplantar keratoderma, acquired palmoplantar keratoderma, hydradenitis suppurativa, dermatitis, ichthyosis vulgaris, hereditary ichthyosis, acquired ichthyosis, actinic keratosis, a keratinization skin disorder, a keratinization mucosal disorder, Gorlin syndrome, nail psoriasis, flexural/inverse psoriasis, non-melanoma skin cancer, Cutaneous T-cell lymphoma and precancerous skin, mucosal and nail lesions, by once or twice daily topical administration to a subject of a composition comprising therapeutically effective amount of tapinarof.

### CROSS REFERENCE TO RELATED APPLICATIONS

This application is a Continuation-in-Part of PCT International Application No. PCT/IL2020/050817, International Filing Date Jul. 23, 2020, claiming the benefit of U.S. Patent Applications Nos. 62/877,966, filed Jul. 24, 2019, and 63/005,353, filed Apr. 5, 2020, which are hereby incorporated by reference.

### FIELD OF THE INVENTION

The present invention, in some embodiments thereof, relates to topical compositions and methods of treatment of skin or mucosal disorders by administering a therapeutically effective amount of tapinarof.

The topical compositions of this invention are useful for the treatment, prevention or alleviation of a skin or mucosal disorder, selected from palmoplantar psoriasis, hereditary palmoplantar keratoderma, acquired palmoplantar keratoderma, hydradenitis suppurativa, dermatitis, ichthyosis vulgaris, hereditary ichthyosis, acquired ichthyosis, actinic keratosis, a keratinization skin disorder, a keratinization mucosal disorder, Gorlin syndrome, nail psoriasis, flexural/inverse psoriasis, non-melanoma skin cancer, Cutaneous T-cell lymphoma and precancerous skin, mucosal and nail lesions.

### BACKGROUND OF THE INVENTION

Tapinarof (3,5-dihydroxy-4-isopropyl-trans-stilbene) is a pharmaceutical active agent investigated for the treatment of atopic dermatitis and psoriasis (Zang Y N, et al., Int J Clin Pharmacol Ther. 2016 February;54(2):87-95). The 3,5-dihydroxy-4-isopropyl-stilbene is also known as benvitimod.

Tapinarof is a first-in-class drug, whose mechanism is not yet fully understood.

Epidermal Growth Factor Receptor (EGFR) inhibitor drugs like erlotinib, gefitinib, osimertinib and brigatinib target the EGFR and are used for the systemic treatment of some forms of cancer (lung, colon).

There is no US-marketed EGFR inhibitor drug for topical use. The EGFR inhibitor erlotinib is sold as oral tablets (Tarceva). Similarly, gefitinib (Iressa), osimertinib (Tigresso) and brigatinib (Alunbrig) are sold as oral tablets.

There is an unmet need for efficient and patient-friendly methods of topical treatment, prevention or alleviation of a skin or mucosal disorder selected from palmoplantar psoriasis, hereditary palmoplantar keratoderma, acquired palmoplantar keratoderma, hydradenitis suppurativa, dermatitis, ichthyosis vulgaris, hereditary ichthyosis, acquired ichthyosis, actinic keratosis, a keratinization skin disorder, a keratinization mucosal disorder, Gorlin syndrome, nail psoriasis, flexural/inverse psoriasis, non-melanoma skin cancer and precancerous skin, mucosal and nail lesions.

### SUMMARY OF THE INVENTION

The present invention discloses topical compositions and methods of treatment of skin or mucosal disorders selected from palmoplantar psoriasis, hereditary palmoplantar keratoderma, acquired palmoplantar keratoderma, hydradenitis suppurativa, dermatitis, ichthyosis vulgaris, hereditary ichthyosis, acquired ichthyosis, actinic keratosis, a keratinization skin disorder, a keratinization mucosal disorder, Gorlin syndrome, nail psoriasis, non-melanoma skin cancer, Cutaneous T-cell lymphoma and precancerous skin, mucosal and nail lesions, by topical administration to a subject of a composition comprising active agent(s) selected from about 0.01% w/w to about 10% w/w or higher tapinarof, from about 0.01% w/w to about 10% w/w at least one EGFR inhibitor and tapinarof-EGFR inhibitor combinations thereof and a carrier suitable for topical administration.

### DETAILED DESCRIPTION OF THE INVENTION

A number of debilitating skin or mucosal disorders such as palmoplantar psoriasis, hereditary palmoplantar keratoderma, acquired palmoplantar keratoderma, hydradenitis suppurativa, dermatitis, ichthyosis vulgaris, hereditary ichthyosis, acquired ichthyosis, actinic keratosis, a keratinization skin disorder, a keratinization mucosal disorder, Gorlin syndrome, nail psoriasis, flexural/inverse psoriasis, non-melanoma skin cancer, Cutaneous T-cell lymphoma and precancerous skin and nail lesions, are still in need for an effective and patient-friendly treatment, such as topical treatment.

The present invention provides topical compositions and topical methods of treatment with a composition comprising active agent(s) selected from tapinarof, a first-in-class drug, at least one Epidermal Growth Factor Receptor inhibitor (henceforth EGFR inhibitor) and tapinarof-EGFR inhibitor combinations.

Tapinarof's efficacy in the treatment of a number of skin conditions, especially atopic dermatitis and plaque psoriasis, has been already proved.

According to Jancin B. (Dermatology News, Nov. 11, 2017), in the above studies, the 1% tapinarof arm showed higher efficacy and had a quicker onset of action than the 0.5% arm or vehicle. The most frequent adverse events associated with tapinarof were folliculitis and contact dermatitis.

Tapinarof, which seems to be a significant advance in psoriasis treatment, presents however higher adverse effects. The rate of treatment-emergent adverse events was higher with tapinarof (93 of 165 [56%]) than with vehicle (34 of 82 [41%]), and the events were mild to moderate in intensity. (Peppers J. et al. J. Amer. Acad. Dermatology, January 2019, vol. 80, Issue 1, pp. 89-98)

There is an unmet need for methods for the treatment of skin or mucosal disorders using tapinarof topical compositions, devoid of serious side-effects.

The present invention solves the aforementioned side-effects, i.a. by encapsulating tapinarof by a process detailed in Examples 1 and 2 (see also U.S. Pat. No. 9,687,465 and published U.S. Patent Application No. 2018147165 (to Sol-Gel Technologies)).

The tapinarof encapsulation process detailed in Examples 1 and 2 allows the use of tapinarof concentrations higher than 2% w/w with minimal or no side-effects.

The selection of an EGFR inhibitor drug as an optional additional active agent in a topical drug is unusual and unexpected, because of the known cutaneous side-effects of this class of active agents.

Treatment with EGFR inhibitors is known to induce cutaneous conditions like acneiform rash, papulopustular rash, abnormal scalp hair growth, abnormal facial hair growth, abnormal hair growth, abnormal eyelash growth, paronychia with or without pyogenic granulomas and telangiectasia.

This is probably one of the reasons that no topical EGFR inhibitor product is marketed so far. A number of clinical studies are underway on the topical treatment or prevention of the EGFR inhibitors-induced cutaneous side-effects, but none on treatment of skin disorders by administration of topical EGFR inhibitors.

It occurred to the present inventors that tapinarof, acting as an anti-inflammatory and EGFR inhibitors, being tyrosine kinase inhibitors and also essential regulators of multiple epidermal functions, as sole active agents but also as tapinarof-EGFR inhibitor combinations, may be used to prevent, cure or alleviate a number of skin or mucosal disorders in which inflammation and/or tyrosine kinase inhibition or epidermal function regulation play a causal mechanistic role. The additive and/or synergistic effect between tapinarof and EGFR enables reducing the amounts of the active agents in the topical combination composition and thus also reduce the cutaneous side-effects. In addition, there are advantages in treating skin disorders by topical instead of systemic administration, thus avoiding systemic side-effects and minimizing, preventing or avoiding cutaneous EGFR inhibitors' side-effects.

An additional advantage of the topical compositions of this invention is the avoidance or minimalization of systemic EGFR inhibitor absorption. The EGFR inhibitor cutaneous side-effects reported in the medical literature are the result of oral (systemic) treatment with EGFR inhibitors. The compositions and methods of treatment of the present invention use topical instead of oral administration, thus avoiding systemic effects, and are therefore expected to present an advantageous cutaneous side-effects profile as compared to the EGFR inhibitor oral products.

Some of the skin or mucosal disorders contemplated for treatment with the methods of this invention are selected from palmoplantar psoriasis, hereditary palmoplantar keratoderma, acquired palmoplantar keratoderma, hydradenitis suppurativa, dermatitis, ichthyosis vulgaris, hereditary ichthyosis, acquired ichthyosis, actinic keratosis, a keratinization skin disorder, a keratinization mucosal disorder, Gorlin syndrome, nail psoriasis, flexural/inverse psoriasis, non-melanoma skin cancer, Cutaneous T-cell lymphoma and precancerous skin, mucosal and nail lesions (see below).

### Palmoplantar Psoriasis (PPP)

Palmoplantar psoriasis is a clinical subtype of psoriasis that characteristically affects the skin of the palms and soles. PPP affects approximately 4% of the patients diagnosed with psoriasis. It features hyperkeratotic, pustular, or mixed morphologies. The condition is chronic in nature and produces significant functional disability (see Miceli A, Schmieder G J. Palmoplantar Psoriasis. [Updated 2019 Jun. 3]. StatPearls [Internet]. Treasure Island (FL): StatPearls Publishing; 2019 January). https://www.ncbi.nlm.nih.gov/books/NBK448142/

### Palmoplantar Keratoderma (PPK)

Palmoplantar keratoderma (also known as palmoplantar keratosis or PPK) appears in three clinically distinct patterns: diffuse, focal and punctate, (Freedberg I M, Fitzpatrick T B (2003). Fitzpatrick's Dermatology in General Medicine (6th ed.), New York; London: McGraw-Hill. p. 505).

The diffuse epidermolytic palmoplantar keratoderma is one of the most common patterns of palmoplantar keratoderma.

The diffuse nonepidermolytic palmoplantar keratoderma is also known as “hereditary palmoplantar keratoderma” and is inherited as an autosomal dominant condition.

Another type of palmoplantar keratoderma—aquagenic keratoderma—is known as “acquired aquagenic palmoplantar keratoderma” (see also Patel S, Zirwas M, English J C (2007). “Acquired palmoplantar keratoderma”. American Journal of Clinical Dermatology. 8 (1): 1-11).

The compositions and methods of treatment of palmoplantar keratoderma of this invention are meant for all types of palmoplantar keratoderma.

### Hidradenitis Suppurativa (HS)

Hidradenitis suppurativa, also known as acne inversa, is a long-term chronic skin disease whose present treatment options are often unsatisfactory. HS has a profound effect on patient's quality of life (QoL). Alavi A. et al., reviewed QoL aspects of this disease in an article titled “Quality-of-Life Impairment in Patients with Hidradenitis Suppurativa” Am J Clin Dermatol. 2015 February;16(1):61-5

The clinical picture of HS includes solitary nodules, diffuse, painful abscesses, malodorous drainage, sinus tract formation and scarring.

The exact cause of hidradenitis suppurativa is still unclear, but it is believed that the underlying mechanism involves dysfunction of the apocrine sweat glands or hair follicles.

There is no cure for HS, but treatments with drugs selected from oral antibiotics, corticosteroid injections, antiandrogen therapy with high dosages of cyproterone acetate and ethynyl estradiol) TNF inhibitors like adalimumab and immunosuppressive drugs have been attempted.

### Dermatitis (Eczema)

Dermatitis is a group of diseases resulting in skin inflammation, itchiness, red skin and rash. The dermatitis group of diseases includes atopic dermatitis (AD), allergic contact dermatitis, irritant contact dermatitis and stasis dermatitis. Atopic dermatitis is the most common type of dermatitis.

### Ichthyosis

Ichthyosis is a rare genetic skin condition, believed to be caused by a mutation in the filaggrin gene (FLG). Ichthyosis vulgaris (the most common form of ichthyosis) is clinically characterized by xerosis, scaling, keratosis pilaris, palmar and plantar hyperlinearity, and a strong association with atopic disorders (Thyssen J. P. et al, British Journal of Dermatology, v. 168, issue 6. pp. 1155-1166).

There are more than 20 types of ichthyosis (Beers, Mark H., MD, and Robert Berkow, MD, editors. “Ichthyosis.” Section 10, Chapter 121).

The compositions and methods of treatment of ichthyosis of this invention are meant for all types of ichthyosis (including but not limited to vulgaris, hereditary, acquired).

### Actinic Keratosis (AK)

AK, also known as senile or solar keratosis, usually appears as a sharply outlined wart-like or keratotic growth, which may develop into a cutaneous horn, and may become malignant; it usually occurs in the middle aged or elderly and is due to excessive exposure to the sun.

One of the possible mechanisms of actinic keratosis is dysregulation of the EGFR signaling, which results in cellular hyperproliferation and defects in differentiation (Joseph SR et al., “Dysregulation of epidermal growth factor receptor in actinic keratosis and squamous cell carcinoma”, Curr Probl. Dermatol. 2015; 46:20-7.

### Keratinization Skin Disorders

This class of skin disorders includes Darier's disease, Hailey-Hailey disease, erythrodermic autosomal recessive lamellar ichthyosis, nonerythrodermic autosomal recessive lamellar ichthyosis, autosomal dominant lamellar ichthyosis, bullous congenital ichthyosiform erythroderma, palmoplantar keratosis, erythrokeratodermia variabilis, verrucous epidermal nevi, pityriasis rubra pilaris, Netherton syndrome, idiopathic vulgaris, ichthyosis vulgaris, monilethrix, keratosis piliaris, bullous ichthyosiform erythroderma, nonbullous congenital ichthyosis, Sjogren-Larsson syndrome, erythrokeratodermica variabilis, hyperkeratosis lenticularis perstans, eythrokeratodermia figurate variabilis, mutilating keratosis of Vohwinkel, Harlequin ichthyosis and Tay's syndrome (International Patent Application PCT/US2009/031101).

A new terminology for the keratinization skin disorders has been recently introduced (see Akiyama M. et al., J Dermatol Sci. 2018 May;90(2):105-111, “Autoinflammatory keratinization diseases: An emerging concept encompassing various inflammatory keratinization disorders of the skin”).

### Keratinization Mucosal Disorders

This class of mucosal (oral, vaginal, anal) disorders includes Lichen Planus, Leukoplakia and Lichen sclerosus.

### Nail Psoriasis

Nail psoriasis affects 10-90% of adult patients with plaque psoriasis, and has been reported in 63-83% of patients with psoriatic arthritis (PsA). In children with psoriasis the prevalence of nail involvement is 32.3%. Nail involvement in psoriatic patients has a significant impact on their quality of life (Reumatologia, 2017,55(1): 44-47).

Nails are skin appendages, so nail psoriasis is a skin disease.

### Flexural/Inverse Psoriasis

Inverse psoriasis is a rare form of psoriasis which is also known as flexural or intertriginous psoriasis. This subtype of psoriasis can occur in any area where two skin surfaces meet. Classically the skin of the groin region, armpits and genitals are affected. In these regions the skin appears red, shiny, and moist, with clear borders, and can sometimes crack in the center.

This rare form of psoriasis accounts for 3-7% of people with psoriasis. A small Chinese study found that the average age of onset for inverse psoriasis is 28.9 years. Occasionally people with another subtype of psoriasis known as pustular psoriasis go on to develop inverse psoriasis. Recent guidelines from the National Psoriasis Foundation recommend the use of low to moderate strength corticosteroids for flare ups of this type of psoriasis and calcipotriene and either tacrolimus or pimecrolimus (e.g. Elidel) for treatment of inverse psoriasis in the long term.

### Precancerous Skin and Nail Lesions

Precancerous lesions are disorders that are highly likely to become malignant. Early diagnosis of precancerous skin, mucosal and nail lesions helps to prevent skin cancers.

A 2012 study (Iran J. Dermatol.2012, 15, 89-94) reported that the most common precancerous skin lesion was actinic keratosis (68.4%) followed by Bowen's disease (7.2%). About 67.5% of the patients were male with a mean age of 61.7 years. Moreover, 53.1% of the patients worked outdoors. The most common site of the lesions was head and neck (83.3%) and 18.7% of lesions were associated with malignancy. The most common pathological form of actinic keratosis was the proliferative type (28.9%).

### Non-melanoma Skin Cancer (NMSC)

Skin cancers include three main types—basal-cell skin cancer (BCC), squamous cell skin cancer (SCC) and melanoma.

The first two types together (BCC and SCC) are known as non-melanoma skin cancers (NMSC).

Cetuximab (Erbitux), an EGFR inhibitor, has been investigated for oral treatment of NMSC (Wollina U., Expert Opinion on Biological Therapy, Vol. 14, 2014—Issue 2).

Uncontrolled signaling from receptor and intracellular tyrosine kinases can lead to numerous proliferative diseases, i.a. cancer (Ben-Bassat H et al. Curr. Pharm Des. 2000 June;6(9):933-42).

### Gorlin Syndrome (NBCCS)

NBCCS (Nevoid Basal-Cell Carcinoma Syndrome) is inter alia a predisposition for BCC caused by a genetic mutation. Oral treatment of NMCS (which includes BCC) with cetuximab (an EGFR inhibitor) has been investigated, but the topical treatment of Gorlin syndrome with topical EGFR inhibitors was never attempted.

### Cutaneous T-Cell Lymphoma

Cutaneous T-cell lymphoma (CTCL) is a rare type of cancer that begins in white blood cells called T cells (T lymphocytes). These cells normally help your body's germ-fighting immune system. In cutaneous T-cell lymphoma, the T cells develop abnormalities that make them attack the skin. Cutaneous T-cell lymphoma can cause rash-like skin redness, slightly raised or scaly round patches on the skin, and, sometimes, skin tumors.

Several types of cutaneous T-cell lymphoma exist. The most common type is mycosis fungoides. Sezary syndrome is a less common type that causes skin redness over the entire body. Some types of cutaneous T-cell lymphoma, such as mycosis fungoides, progress slowly and others are more aggressive. Cutaneous T-cell lymphoma is one of several types of lymphoma collectively called non-Hodgkin's lymphoma.

In some embodiments, the EGFR inhibitor in the present invention is selected from gefitinib, erlotinib, lapatinib, cetuximab, panitumumab, vandetanib, necitumumab, osimertinib and combinations thereof.

The present invention provides novel methods of treatment of skin or mucosal disorders selected from palmoplantar psoriasis, hereditary palmoplantar keratoderma, acquired palmoplantar keratoderma, hydradenitis suppurativa, dermatitis, ichthyosis vulgaris, hereditary ichthyosis, acquired ichthyosis, actinic keratosis, a keratinization skin disorder, a keratinization mucosal disorder, Gorlin syndrome, nail psoriasis, flexural/inverse psoriasis, non-melanoma skin cancer, Cutaneous T-cell lymphoma and precancerous skin, mucosal and nail lesions by topical administration of a composition comprising active agents selected from tapinarof, at least one EGFR inhibitor and tapinarof-EGFR inhibitor(s) combinations.

In some embodiments there is provided a method of treatment of a skin or mucosal disorder in which epidermal function regulation or tyrosine kinase inhibition play a causal mechanistic role, by topical administration of a therapeutically effective amount of at least one EGFR inhibitor.

According to some embodiments, there is provided a topical composition for the treatment of a skin or mucosal disorder selected from palmoplantar psoriasis, hereditary palmoplantar keratoderma, acquired palmoplantar keratoderma, hydradenitis suppurativa, dermatitis, ichthyosis vulgaris, hereditary ichthyosis, acquired ichthyosis, actinic keratosis, a keratinization skin disorder, a keratinization mucosal disorder, Gorlin syndrome, nail psoriasis, flexural/inverse psoriasis, non-melanoma skin cancer, Cutaneous T-cell lymphoma and precancerous skin, mucosal and nail lesions, wherein said composition comprising from about 0.01% to about 10% or higher tapinarof and a carrier suitable for topical administration. In another embodiment, said composition comprises from about 0.01% to about 1% w/w, from about 1% to about 3% w/w, from about 3% to about 5% w/w, from about 5% to about 10% w/w tapinarof and a carrier suitable for topical administration. In another embodiment, the composition comprises 5% w/w tapinarof and a carrier suitable for topical administration. In another embodiment, the composition comprises 10% w/w tapinarof and a carrier suitable for topical administration.

According to some embodiments, there is provided a topical composition for the treatment, prevention or alleviation of nail psoriasis, wherein said composition comprises between 5% to about 10% w/w tapinarof. In another embodiment, the composition comprises 5% w/w tapinarof. In another embodiment, the composition comprises 10% w/w tapinarof. In another embodiment, the composition for use in treating, preventing or alleviating nail psoriasis comprises a solution of between 5% to 10% w/w tapinarof and a carrier suitable for topical administration.

According to some embodiments, there is provided a topical composition for the treatment of a skin or mucosal disorder selected from palmoplantar psoriasis, hereditary palmoplantar keratoderma, acquired palmoplantar keratoderma, hydradenitis suppurativa, dermatitis, ichthyosis vulgaris, hereditary ichthyosis, acquired ichthyosis, actinic keratosis, a keratinization skin disorder, a keratinization mucosal disorder, Gorlin syndrome, nail psoriasis, flexural/inverse psoriasis, non-melanoma skin cancer, Cutaneous T-cell lymphoma and precancerous skin, mucosal and nail lesions, wherein said composition comprising from about 0.01% to about 10% w/w, from about 0.01% to about 1% w/w, from about 1% to about 3% w/w, from about 3% to about 5% w/w, or from about 5% to about 10% w/w at least one EGFR inhibitor selected from erlotinib, gefitinib, lapatinib, cetuximab, panitumumab, vandetanib, necitumumab, osimertinib and combinations thereof and a carrier suitable for topical administration.

According to some embodiments, there is provided a topical composition for the treatment of a skin or mucosal disorder selected from palmoplantar psoriasis, hereditary palmoplantar keratoderma, acquired palmoplantar keratoderma, hydradenitis suppurativa, dermatitis, ichthyosis vulgaris, hereditary ichthyosis, acquired ichthyosis, actinic keratosis, a keratinization skin disorder, a keratinization mucosal disorder, Gorlin syndrome, nail psoriasis, flexural/inverse psoriasis, non-melanoma skin cancer, Cutaneous T-cell lymphomaand precancerous skin, mucosal and nail lesions, wherein said composition comprising from about 0.01% to about 10% w/w, from about 0.01% to about 1%, from about 1% to about 3%, from about 3% to about 5% w/w or from about 5% to about 10% w/w tapinarof, from about 0.01% to about 10% w/w from about 0.01% to about 1% w/w, from about 1% to about 3% w/w, from about 3% to about 5% w/w, or from about 5% to about 10% w/w at least one EGFR inhibitor selected from erlotinib, gefitinib, lapatinib, cetuximab, panitumumab, vandetanib, necitumumab, osimertinib and combinations thereof and a carrier suitable for topical administration.

The above topical composition may further comprise at least one additional active agent.

In some embodiments, there is provided the above topical composition, further comprising at least one additional active agent selected from menadione, ketoconazole, dapsone, cevimeline, spironolactone, tretinoin, pimecrolimus, tetracycline, a sunscreen, doxycycline, epidermal growth factor (EGF), lycopene, threolone, synthomycine, erythromycin, Vitamin K3 and combinations thereof, in a concentration of from about 0.01% to about 5% w/w, from about 0.01% to about 1%, from about 1% to about 3% or from about 3% to about 5% w/w.

Some of the above additional active agents, selected from menadione, ketoconazole, dapsone, cevimeline, spironolactone, tretinoin, pimecrolimus, tetracycline, a sunscreen, doxycycline, epidermal growth factor (EGF), lycopene, threolone, synthomycine, erythromycin, Vitamin K3 and combinations thereof, play the role of avoiding, preventing or alleviating the EGFR inhibitor cutaneous side-effects.

According to some embodiments, there is provided a method of treatment of a skin or mucosal disorder selected from the group consisting of palmoplantar psoriasis, hereditary palmoplantar keratoderma, acquired palmoplantar keratoderma, hydradenitis suppurativa, dermatitis, ichthyosis vulgaris, hereditary ichthyosis, acquired ichthyosis, actinic keratosis, a keratinization skin disorder, a keratinization mucosal disorder, Gorlin syndrome, nail psoriasis, flexural/inverse psoriasis, non-melanoma skin cancer, Cutaneous T-cell lymphoma and precancerous skin, mucosal and nail lesions by topical administration to a subject in need thereof a therapeutically effective amount of a composition comprising from about 0.01% to about 10% w/w, from about 0.01% to about 1% w/w, from about 1% to about 3% w/w, from about 3% to about 5% w/w or from about 5% to about 10% w/w at least one EGFR inhibitor selected from the group consisting of erlotinib, gefitinib, lapatinib, cetuximab, panitumumab, vandetanib, necitumumab, osimertinib and combinations thereof and at least one additional active agent selected from menadione, ketoconazole, dapsone, cevimeline, spironolactone, tretinoin, pimecrolimus, tetracycline, a sunscreen, doxycycline, epidermal growth factor (EGF), lycopene, threolone, synthomycine, erythromycin, Vitamin K3 and combinations thereof, in a concentration of from about 0.01% to about 3% w/w, from about 0.01% to about 1% w/w, from about 1% to about 3% w/w or from about 3% to about 5% w/w.

In some embodiments, there is provided a method of treatment comprising once or twice daily topical application of therapeutically effective amounts of the said combination composition or two separate topical compositions to the skin portion of the subject affected by the said skin or mucosal disorder until the skin or mucosal disorder is cured, prevented or alleviated or according to doctor's instructions.

In some other embodiments, the EGFR inhibitor in any of the methods and compositions of this invention is erlotinib.

Typical formulations for topical administration include creams, ointments, gels, sprays, lotions, foams, shampoos and patches.

According to some embodiments, the topical compositions of this invention are selected from a cream, an ointment, a gel, a lotion, a spray, a shampoo, a patch and a foam.

The compositions, combinations and articles of manufacture of this invention can be administered using a variety of routes such as topical application or transdermal application. The preferred route is the topical route and the preferred formulations are the cream, the lotion, the gel, the shampoo and the foam.

The active agents in the combination compositions are included in an amount effective for treating, preventing or alleviating the inflammatory skin condition or specifically the acne or rosacea symptoms. The concentration of the active agents in the composition will depend on absorption, inactivation, excretion rates of the active agent, the synergistic or additive effects, the dosage schedule, and amount administered as well as other factors known to those of skill in the art.

Typically, the dosages and concentrations of the active agents in the combination composition of this invention will be lower, typically at least about or at 5 to 10% lower but up to about or at 15, 20, 25, 30, 35, 40, 50, 90 or 95% lower than the amount of same active agents in the marketed single drug currently administered or being developed for the treatment of the skin condition. The dosage and regimen of administration may be determined by dose finding studies, as known in the art.

Exemplary strengths and concentrations of tapinarof in the topical compositions comprising tapinarof of this invention are 0.01%, 0.03%, 0.05%, 0.08%, 0.1%, 0.25%, 0.5%, 1%, 2%, 3%, 4% , 5% , 6%, 7%, 8%, 9% or 10% w/w. Typical strengths in the topical compositions of this invention are about 0.1%, about 1%, about 2% or about 3% w/w tapinarof, about 5% tapinarof, about 10% tapinarof or higher. In another embodiment, the concentration of tapinarof is between 0.01% and 10% w/w; between 0.1% and 1.5% w/w; between 0.5%-2% w/w; between 1% and 5% w/w or between 5% to 10% w/w.

Exemplary strengths and concentrations of the least one EGFR inhibitor in the topical compositions of this invention comprising an EGFR inhibitor from about 0.01% to about 10% w/w, from about 0.01% to about 1% w/w, from about 1% to about 3% w/w, from about 3% to about 5% w/w or from about 5% to about 10% w/w. Typical strengths in the topical combination compositions of this invention are 0.1%, 0.25%, 0.5% or 1% w/w.

Exemplary strengths and concentrations of the least one additional active agent in the compositions of this invention, selected from menadione, ketoconazole, dapsone, cevimeline, spironolactone, tretinoin, pimecrolimus, tetracycline, a sunscreen, doxycycline, epidermal growth factor (EGF), lycopene, threolone, synthomycine, erythromycin, Vitamin K3 and combinations thereof in the topical combination compositions are 0.1%, 0.25%, 0.5%, 1%, 2%, 3%, 4% and 5% w/w. Typical strengths in the topical combination compositions of this invention are 1%, 2% or 3% w/w.

The frequency of administration can be determined empirically.

Exemplary frequencies are once daily, twice daily, weekly, bi-weekly or monthly. Typical administration frequencies of the topical combination compositions of this invention are once daily and twice daily.

Dosage frequencies can be gradually decreased over time and maintained at a steady dose suitable for long-term—six months, 1 year, 5 years, 10 years or more, up to lifelong administration to control the symptoms of a skin or mucosal disorder. For example, dosage administration can begin at from twice a day, to once a day, to two times a week, to once a week, to once every two weeks or less frequent than once every two weeks.

Pharmaceutical carriers or vehicles suitable for preparation of the compositions provided herein include any such carriers known to those skilled in the art to be suitable for the particular mode of administration.

The resulting composition may be a lotion, a solution, a suspension, an emulsion or the like and is formulated as creams, gels, ointments, emulsions, solutions, elixirs, lotions, suspensions, tinctures, pastes, foams, aerosols, sprays, patches, foams, sebum control products or any other formulation suitable for topical administration. The preferred compositions are the cream, the lotion, the gel and the foam.

Pharmaceutical carriers or vehicles suitable for administration of the compounds provided herein include any such carriers known to those skilled in the art to be suitable for the particular mode of administration.

Sebum control products may include ingredients selected from azelaic acid, salicylic acid, sulfur, nicotinamide, L-carnitine and combinations thereof.

In addition, tapinarof or the at least one EGFR inhibitor active agent may be formulated as the sole pharmaceutically active agent in the composition or may be combined. The active agents are included in the carrier in an amount sufficient to exert a therapeutically useful effect i.e., amelioration of the symptoms of a skin or mucosal disorder, with minimal or no toxicity or other side effects.

Generally, emollient or lubricating vehicles that help hydrate the skin are more preferred than volatile vehicles, such as ethanol, that dry the skin. Examples of suitable bases or vehicles for preparing compositions for use with human skin are petrolatum, petrolatum plus volatile silicones, lanolin, cold cream and hydrophilic ointment.

Suitable pharmaceutically and dermatologically acceptable vehicles for topical application include lotions, creams, foams, solutions, gels, patches and the like. Generally, the vehicle is either organic in nature or an aqueous emulsion and capable of accommodating the selected active agent(s), which may be micronized, dispersed, suspended or dissolved therein. The vehicle may include pharmaceutically-acceptable emollients, moisturizers, including lactic acid, ammonium lactate and urea, skin penetration enhancers, coloring agents, fragrances, emulsifiers, thickening agents, vegetable oils, essential oils, zinc oxide and solvents.

### Methods of Treatment

According to an aspect of the invention, there is provided a method of treatment, prevention or alleviation of a skin or mucosal disorder selected from palmoplantar psoriasis, hereditary palmoplantar keratoderma, acquired palmoplantar keratoderma, hydradenitis suppurativa, ichthyosis vulgaris, hereditary ichthyosis, acquired ichthyosis, actinic keratosis, a keratinization skin disorder, a keratinization mucosal disorder, Gorlin syndrome, nail psoriasis, flexural/inverse psoriasis, non-melanoma skin cancer, Cutaneous T-cell lymphoma and precancerous skin, mucosal and nail lesions, by topical administration to a subject in need thereof a therapeutically effective amount of the composition and combinations thereof and a carrier suitable for topical administration, wherein the composition is formulated in a dosage form selected from a cream, a gel, an ointment, an emulsion, a solution, a suspension, an elixir, a lotion, a tincture, a paste, a foam, an aerosol, a spray, a patch, a transdermal patch and a pre-filled applicator syringe.

In another aspect of this invention the skin or mucosal disorder is palmoplantar psoriasis. In another aspect of this invention the skin or mucosal disorder is hereditary palmoplantar keratoderma. In another aspect of this invention the skin or mucosal disorder is acquired palmoplantar keratoderma. In another aspect of this invention the skin or mucosal disorder is hydradenitis suppurativa. In another aspect of this invention the skin or mucosal disorder is ichthyosis vulgaris. In another aspect of this invention the skin or mucosal disorder is hereditary ichthyosis. In another aspect of this invention the skin or mucosal disorder is acquired ichthyosis. In another aspect of this invention the skin or mucosal disorder is actinic keratosis. In another aspect of this invention the skin or mucosal disorder is a keratinization skin disorder. In another aspect of this invention the skin or mucosal disorder is a keratinization mucosal disorder. In another aspect of this invention the skin or mucosal disorder is Gorlin syndrome. In another aspect of this invention the skin or mucosal disorder is nail psoriasis. In another aspect of this invention the skin or mucosal disorder is flexural/inverse psoriasis. In another aspect of this invention the skin or mucosal disorder is non-melanoma skin cancer. In another aspect of this invention the skin or mucosal disorder is Cutaneous T-cell lymphoma. In another aspect of this invention the skin or mucosal disorder is precancerous skin. In another aspect of this invention the skin or mucosal disorder is mucosal and nail lesions.

In some embodiments, the effective amount is a therapeutically effective amount of a composition comprising tapinarof, EGFR, combination thereof and optionally additional active agents, namely an amount which will cure, treat, prevent or alleviate a skin or mucosal disorder.

In some other embodiments, the co-administration may be made either by administration of a single combination composition, or alternatively by separate administration of a first composition comprising one of the active agents (e.g. tapinarof or at least one EGFR inhibitor) and a carrier suitable for topical administration and a second composition comprising the other active agent(s) and a carrier suitable for topical administration.

In some embodiment, this invention provides a method of treating, preventing or alleviating nail psoriasis comprises administering between 5% to about 10% w/w tapinarof. In another embodiment, the method comprises administering 5% w/w tapinarof. In another embodiment, the method comprises administering 10% w/w tapinarof. In another embodiment, the method for treating, preventing or alleviating nail psoriasis comprises administering a solution of between 5% to 10% w/w tapinarof and a carrier suitable for topical administration.

### Regimen of Administration of the Topical Combination Compositions

Therapeutically effective concentrations of active agents in the compositions of this invention for treatment, prevention or amelioration of the symptoms manifested by a skin or mucosal disorder are determined by empirical methods known in the art.

The frequency of administration can be determined empirically. Exemplary frequencies are once daily, twice daily, weekly, bi-weekly or monthly.

Typical administration frequencies of the topical combination compositions of this invention are once daily and twice daily.

Dosage frequencies can be gradually decreased over time and maintained at a steady dose suitable for long-term—six months, 1 year, 5 years, 10 years or more, up to lifelong administration to control the symptoms of the skin or mucosal disorder. For example, dosage administration can begin at from twice a day, to once a day, to two times a week, to once a week, to once every two weeks or less frequent than once every two weeks.

### Kits

Kits containing the compositions of this invention, optionally including instructions for administration are provided. Additionally, provided herein are kits containing the above-described combinations and optionally instructions for administration by topical, transdermal, or other routes, depending on the single composition or two separate compositions to be delivered.

The compositions provided herein can be packaged as articles of manufacture containing packaging material, a composition provided herein, and a label that indicates that the composition is for treating a skin or mucosal disorder and is formulated for topical delivery.

The articles of manufacture provided herein contain packaging materials. Packaging materials for use in packaging pharmaceutical products are well known to those of skill in the art.

Examples of pharmaceutical packaging materials include, but are not limited to bottles, tubes, containers, application syringes or dual chamber application syringes and any packaging material suitable for the selected formulation and intended mode of administration and treatment.

As EGFR inhibitors in general and erlotinib in particular are poorly soluble, the compositions of this invention need to comprise a high EGFR inhibitor concentration of up to 10% w/w. The compositions are in the form of partly solubilized suspensions and comprise organic solvents and solubility enhancers.

### EMBODIMENTS

In some embodiments, there is provided a topical composition for the treatment, prevention or alleviation of a skin or mucosal disorder, selected from palmoplantar psoriasis, hereditary palmoplantar keratoderma, acquired palmoplantar keratoderma, hydradenitis suppurativa, ichthyosis vulgaris, hereditary ichthyosis, acquired ichthyosis, actinic keratosis, a keratinization skin disorder, a keratinization mucosal disorder, Gorlin syndrome, nail psoriasis, flexural/inverse psoriasis, non-melanoma skin cancer, Cutaneous T-cell lymphoma and precancerous skin, mucosal and nail lesions, comprising from about 0.01% to about 10% w/w tapinarof and a carrier suitable for topical administration. In another embodiment, the topical composition comprises from about 0.01% to about 1% w/w, from about 1% to about 3% w/w, from about 3% to about 5% w/w or from about 5% to about 10% tapinarof and a carrier suitable for topical administration.

In some embodiments, there is provided a topical composition for the treatment, prevention or alleviation of a skin or mucosal disorder, selected from palmoplantar psoriasis, hydradenitis suppurativa, dermatitis, actinic keratosis, Gorlin syndrome, nail psoriasis, flexural/inverse psoriasis, non-melanoma skin cancer, Cutaneous T-cell lymphoma and precancerous skin, mucosal and nail lesions, comprising from about 0.01% to about 10% w/w, from about 0.01% to about 1% w/w, from about 1% to about 3% w/w, from about 0.01% to about 1% w/w, from about 1% to about 3% w/w, from about 3% to about 5% w/w or from about 5% to about 10% w/w at least one EGFR inhibitor selected from erlotinib, gefitinib, lapatinib, cetuximab, panitumumab, vandetanib, necitumumab, osimertinib and combinations thereof and a carrier suitable for topical administration.

In some embodiments, there is provided a topical composition for the treatment, prevention or alleviation of a skin or mucosal disorder, selected from palmoplantar psoriasis, hereditary palmoplantar keratoderma, acquired palmoplantar keratoderma, hydradenitis suppurativa, dermatitis, ichthyosis vulgaris, hereditary ichthyosis, acquired ichthyosis, actinic keratosis, a keratinization skin disorder, a keratinization mucosal disorder, Gorlin syndrome, nail psoriasis, flexural/inverse psoriasis, non-melanoma skin cancer, Cutaneous T-cell lymphoma and precancerous skin, mucosal and nail lesions, comprising from about 0.01% to about 10% w/w, from about 0.01% to about 1% w/w, from about 1% to about 3% w/w, from about 3% to about 5% w/w, or from about 5% to about 10% w/w tapinarof, from about 0.01% to about 10% w/w, from about 0.01% to about 1% w/w, from about 0.01% to about 1% w/w, from about 1% to about 3% w/w, from about 3% to about 5% w/w or from about 5% to about 10% w/w at least one EGFR inhibitor selected from erlotinib, gefitinib, lapatinib, cetuximab, panitumumab, vandetanib, necitumumab, osimertinib and combinations thereof and a carrier suitable for topical administration.

In some embodiments, there is provided a topical composition for the treatment, prevention or alleviation of a skin or mucosal disorder, selected from palmoplantar psoriasis, hereditary palmoplantar keratoderma, acquired palmoplantar keratoderma, hydradenitis suppurativa, dermatitis, ichthyosis vulgaris, hereditary ichthyosis, acquired ichthyosis, actinic keratosis, a keratinization skin disorder, a keratinization mucosal disorder, Gorlin syndrome, nail psoriasis, flexural/inverse psoriasis, non-melanoma skin cancer, Cutaneous T-cell lymphoma and precancerous skin, mucosal and nail lesions, comprising from about 5% to about 10% w/w tapinarof, and from about 0.01% to about 1% w/w, from about 0.01% to about 1% w/w, from about 1% to about 3% w/w, from about 3% to about 5% w/w or from about 5% to about 10% w/w at least one EGFR inhibitor selected from erlotinib, gefitinib, lapatinib, cetuximab, panitumumab, vandetanib, necitumumab, osimertinib and combinations thereof and a carrier suitable for topical administration.

In some embodiments, there is provided a composition comprising tapinarof or tapinarof and at least one EGFR inhibitor of this invention, wherein tapinarof is encapsulated using the process detailed in Example 1 or 2.

In some embodiments, there is provided a composition of this invention comprising tapinarof or at least one EGFR inhibitor or a tapinarof-EGFR inhibitor combination, further comprising a moisturizer, urea, ammonium lactate or combinations thereof.

In some embodiments, there is provided a composition comprising tapinarof or at least one EGFR inhibitor or a tapinarof-EGFR inhibitor combination, further comprising a penetration enhancer.

In some embodiments, there is provided the above composition comprising tapinarof or at least one EGFR inhibitor or a tapinarof-EGFR inhibitor combination, and a penetration enhancer, wherein the penetration enhancer is selected from DMSO, propylene glycol, dimethyl isosorbide, isopropyl myristate and combinations thereof.

In some embodiments, there is provided an EGFR inhibitor-containing composition of this invention, further comprising from about 0.01% to about 5% w/w, from about 0.01% to about 1% w/w, from about 1% to about 3% w/w, from about 3% to about 5% w/w of an ingredient for the alleviation of the EGFR cutaneous side-effects, selected from menadione, ketoconazole, dapsone, cevimeline, spironolactone, tretinoin, pimecrolimus, tetracycline, a sunscreen, doxycycline, epidermal growth factor (EGF), lycopene, threolone, synthomycine, erythromycin, Vitamin K3 and combinations thereof.

In some embodiments, there is provided a composition of this invention, comprising tapinarof or at least one EGFR inhibitor or a tapinarof-EGFR inhibitor combination, wherein said composition is formulated as a cream, an ointment, a gel, a lotion, a shampoo, a spray, a patch or a foam.

In some embodiments, there is provided a method of treatment, prevention or alleviation of a skin or mucosal disorder selected from palmoplantar psoriasis, hereditary palmoplantar keratoderma, acquired palmoplantar keratoderma, hydradenitis suppurativa, ichthyosis vulgaris, hereditary ichthyosis, acquired ichthyosis, actinic keratosis, a keratinization skin disorder and a keratinization mucosal disorder, Gorlin syndrome, nail psoriasis, flexural/inverse psoriasis, non-melanoma skin cancer, Cutaneous T-cell lymphomaand precancerous skin, mucosal and nail lesions by topical administration to a subject in need thereof of a therapeutically effective amount of a composition of this invention comprising from about 0.01% to about 10% w/w, 0.01% to about 1% w/w, from about 1% to about 3% w/w, from about 3% to about 5% w/w or from about 5% to about 10% w/w tapinarof and a carrier suitable for topical administration.

In some embodiments, there is provided a method of treatment, prevention or alleviation of a skin or mucosal disorder selected from palmoplantar psoriasis, hydradenitis suppurativa, dermatitis, actinic keratosis, nail psoriasis, flexural/inverse psoriasis, Gorlin syndrome, non-melanoma skin cancer, Cutaneous T-cell lymphoma and precancerous skin, mucosal and nail lesions, by topical administration to a subject in need thereof a therapeutically effective amount of the composition comprising from about 0.01% to about 10% w/w, from about 0.01% to about 1% w/w, from about 1% to about 3% w/w, from about 3% to about 5% w/w or from about 5% to about 10% w/w at least one EGFR inhibitor selected from erlotinib, gefitinib, lapatinib, cetuximab, panitumumab, vandetanib, necitumumab, osimertinib and combinations thereof and a carrier suitable for topical administration.

In some embodiments, there is provided a method of treatment, prevention or alleviation of a skin or mucosal disorder selected from palmoplantar psoriasis, hereditary palmoplantar keratoderma, acquired palmoplantar keratoderma, hydradenitis suppurativa, dermatitis, ichthyosis vulgaris, hereditary ichthyosis, acquired ichthyosis, actinic keratosis, a keratinization skin disorder and a keratinization mucosal disorder, Gorlin syndrome, nail psoriasis, flexural/inverse psoriasis, non-melanoma skin cancer, Cutaneous T-cell lymphoma and precancerous skin, mucosal and nail lesions by topical administration to a subject in need thereof a therapeutically effective amount of the composition comprising from about 0.01% to about 10% w/w, from about 0.01% to about 1% w/w, from about 1% to about 3% w/w, from about 3% to about 5% w/w or from about 5% to about 10% w/w tapinarof, from about 0.01% to about 10% w/w, from about 0.01% to about 1% w/w, from about 0.01% to about 1% w/w, from about 1% to about 3% w/w, from about 3% to about 5% w/w or from about 5% to about 10% w/w at least one EGFR inhibitor selected from erlotinib, gefitinib, lapatinib, cetuximab, panitumumab, vandetanib, necitumumab, osimertinib and combinations thereof and a carrier suitable for topical administration.

In some embodiments, there is provided a method of treatment, prevention or alleviation of a skin or mucosal disorder selected from palmoplantar psoriasis, hereditary palmoplantar keratoderma, acquired palmoplantar keratoderma, hydradenitis suppurativa, dermatitis, ichthyosis vulgaris, hereditary ichthyosis, acquired ichthyosis, actinic keratosis, a keratinization skin disorder and a keratinization mucosal disorder, Gorlin syndrome, nail psoriasis, flexural/inverse psoriasis, non-melanoma skin cancer, Cutaneous T-cell lymphoma and precancerous skin, mucosal and nail lesions by topical administration to a subject in need thereof, a composition comprising from about from about 5% to about 10% w/w tapinarof, and from about 0.01% to about 10% w/w , from about 0.01% to about 1% w/w, from about 0.01% to about 1% w/w, from about 1% to about 3% w/w, from about 3% to about 5% w/w or from about 5% to about 10% w/w at least one EGFR inhibitor selected from erlotinib, gefitinib, lapatinib, cetuximab, panitumumab, vandetanib, necitumumab, osimertinib and combinations thereof and a carrier suitable for topical administration.

In some embodiments, there is provided any one of the aforementioned methods of treatment of this invention, wherein the skin disorder is selected from palmoplantar psoriasis, hereditary palmoplantar keratoderma and acquired palmoplantar keratoderma.

In some embodiments, there is provided any one of the aforementioned methods of treatment of this invention, wherein tapinarof and said at least one EGFR inhibitor exhibit an additive or synergistic effect.

In some embodiments, there is provided any one of the aforementioned methods of treatment of this invention, wherein the skin disorder is palmoplantar psoriasis.

In some embodiments, there is provided any one of the aforementioned methods of treatment of this invention, wherein the skin disorder is palmoplantar keratoderma.

In some embodiments, there is provided any one of the aforementioned methods of treatment of this invention, wherein the skin or mucosal disorder is selected from a keratinization skin disorder and a keratinization mucosal disorder.

In some embodiments, there is provided any one of the aforementioned methods of treatment of this invention, wherein it comprises once or twice daily topical application of therapeutically effective amounts of the said composition to the skin portion of the subject affected by the said skin or mucosal disorder until the skin or mucosal disorder is cured, prevented or alleviated or according to doctor's instructions.

In some embodiments, there is provided a method or composition of any one of the EGFR inhibitor-comprising compositions of this invention, wherein the EGFR inhibitor is erlotinib.

In some embodiments the compositions, kits and methods of this invention are for treatment, prevention or alleviation of a skin or mucosal disorder. In another embodiment, the skin or mucosal disorder is palmoplantar psoriasis. In another embodiment the skin or mucosal disorder is hereditary palmoplantar keratoderma. In another embodiment the skin or mucosal disorder is acquired palmoplantar keratoderma. In another embodiment the skin or mucosal disorder is hydradenitis suppurativa. In another embodiment the skin or mucosal disorder is ichthyosis vulgaris. In another embodiment the skin or mucosal disorder is hereditary ichthyosis. In another embodiment the skin or mucosal disorder is acquired ichthyosis. In another embodiment the skin or mucosal disorder is actinic keratosis. In another embodiment the skin or mucosal disorder is a keratinization skin disorder. In another embodiment the skin or mucosal disorder is a keratinization mucosal disorder. In another embodiment the skin or mucosal disorder is Gorlin syndrome. In another embodiment the skin or mucosal disorder is nail psoriasis. In another embodiment the skin or mucosal disorder is flexural/inverse psoriasis. In another embodiment the skin or mucosal disorder is non-melanoma skin cancer. In another embodiment the skin or mucosal disorder is Cutaneous T-cell lymphoma. In another embodiment the skin or mucosal disorder is precancerous skin. In another embodiment the skin or mucosal disorder is mucosal and nail lesions.

### Definitions

As used herein, the terms “pharmaceutically active agent” or “active agent” or “active pharmaceutical ingredient” or “API” are interchangeable and mean the ingredient is a pharmaceutical drug which is biological active and is regulatory approved or approvable as such.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “10 μm” is intended to mean “about 10 μm”.

As used herein, numerical ranges preceded by the term “about” should not be considered to be limited to the recited range. Rather, numerical ranges preceded by the term “about” should be understood to include a range accepted by those skilled in the art for any given element in microcapsules or formulations according to the present invention.

The term “about” as used herein means within an acceptable error range for a particular value as determined by one of ordinary skill in the art, which will depend in part on how the value is measured or determined, i.e., the limitations of the measurement system. For example, “about” can mean a range of up to 10%, more preferably up to 5%, and still more preferably up to 1% of a given value. Where particular values are described in the application and claims, unless otherwise stated, the meaning of the term “about” is within an acceptable error range for the particular value.

The terms “comprise”, “comprising”, “includes”, “including”, “having” and their conjugates mean “including but not limited to”.

The term “consisting of” means “including and limited to”.

The term “consisting essentially of” means that the composition, method or microcapsules may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

### EXAMPLES

In the examples below, all % values referring to a solution are in (w/w).

All % values, referring to dispersions (suspensions) are in (w/w).

Unless otherwise indicated, all solutions used in the example below refer to an aqueous solution of the indicated ingredient.

### Example 1

### Preparation of Encapsulated Tapinarof Dispersed in Water (15% E-Tapinarof)

### Preparation of Tapinarof Dispersion and Acid Cocktail

Tapinarof dispersion is prepared by mixing 378 grams of CTAC CT-429 (Cetrimonium Chloride 30%), 6,756 grams of tapinarof, and 18,855 grams water under high shear. The dispersion is homogenized for 60 min at 33° C. (no more than 45° C.).

An acid cocktail is prepared using 1013 grams Hydrochloric Acid (37%), 215.3 grams anhydrous Citric Acid, 322.3 grams Lactic Acid (90%), and 1632 grams water.

### a) Coating Cycle

The coating cycle is started by adding 953 grams sodium silicate solution extra pure (28%) to the tapinarof dispersion prepared in step a) under high shear, followed by adding the acid cocktail prepared in step (a) and followed by adding 1675 grams Polyquarternium-7 (3%) solution to the mixture. The cycle is repeated another 5 times. After the 6 cycles, the pH of the mixture is adjusted to 5.0 using the acid cocktail, and water is added to complete the total weight of the mixture to 45 kilograms.

The composition of the final E-tapinarof water suspension product is shown in Table 1.

TABLE 1
Composition of the encapsulated tapinarof 15% water suspension

| | % w/w of ingredient in the |
|---|---|
| Ingredient | suspension |
| Polyquarternium-7 | 5.6 |
| Hydrochloric Acid 37% | 2.0 |
| Citric Acid, Anhydrous | 0.4 |
| Lactic Acid | 0.6 |
| Silicone Dioxide | 3.4 |
| Sodium Hydroxide | 1.4 |
| Cetrimonium Chloride | 0.84 |
| Tapinarof | 15.00 |
| Sterile Water for Irrigation | Up to 100% |

### Example 2

### Preparation of Encapsulated Tapinarof Dispersed in Oil (3.06% E-Tapinarof)

### a) Oil Phase

45.9 grams of tapinarof are mixed in 129.3 grams of Squalane. 86.16 grams of Tetraethoxysilane (TEOS) are added, and the resulted mixture was milled at 5000 rpm in a ball mill for 10 minutes with an upper propeller mixer at a speed of 250 rpm for 7 minutes, followed by 400 rpm for 3 minutes. 140.4 grams of milled tapinarof in oil is aliquoted out and then heated to 60 ° C. 9.0 grams of Beeswax are added and melted in the oil phase.

### b) Water Phase

3.3 grams CTAC (Cetrimonium Chloride) are dissolved in 490.0 g water at 60° C. Unless indicated otherwise, in all examples described herein, the term “water” refers to sterile water for irrigation (USP).

### c) Core-Shell Step

124.5 grams of the oil phase prepared in step (a) is added to the water phase and homogenized at 4000 rpm for 1 minute. 17.9 grams of sodium silicate extra pure solution (28%) are added to the emulsion. The pH of the emulsion is adjusted to 4.0 using HCl 5N solution. Water is added to complete the total weight of the mixture to 650 grams. The suspension is then stirred for 17 hours at 25° C. for the TEOS hydrolysis to be completed. The composition of the final encapsulated tapinarof water suspension product is shown in Table 2.

TABLE 2
Composition of the encapsulated tapinarof 3.06% water suspension

| | % of pure ingredient in the |
|---|---|
| Ingredient | suspension |
| Beeswax | 1.15 |
| Squalane | 8.62 |
| TEOS | 5.74 |
| Tapinarof | 3.06 |
| Cetrimonium Chloride | 0.15 |
| Sodium hydroxide | 0.74 |
| Hydrochloric acid | 0.40 |
| Sterile Water for Irrigation | Up to 100% |

### Example 3

### Preparation of Tapinarof Solution (5% w/w or 10% w/w)

| | | Tapinarof 10% solution | Tapinarof 5% solution |
|---|---|---|---|
| # | Excipient | w/w % | w/w % |
| 1. | Ethanol Absolute | 57.00 | 62% |
| 2. | Diethyl Sebacate | 20.00 | 20.00 |
| 3. | Transcutol | 12.00 | 12.00 |
| 4. | BHT | 0.10 | 0.10 |
| 5. | Tapinarof | 10.00 | 5.00 |
| 6. | Ethanol Absolute | q.s to 100 | q.s to 100 |

Into a glass beaker, ethanol absolute, diethyl sebacate and transcutol were weighted. The solvents were mixed with magnetic stirrer until a homogenous clear solution was obtained.

Into the solution, BHT was added and the mixing was continued until a clear solution free from particles was obtained.

Then, the beaker was transferred into a yellow light hood and covered with aluminum foil. Tapinarof was added gradually while the mixing was continued for about 30 min to 1 hr until clear yellow solution free from particles was obtained.

The batch was completed with ethanol absolute and was mixed until a homogenous clear solution was obtained.

What is claimed is:

1. A method of treatment, prevention or alleviation of a skin or mucosal disorder selected from palmoplantar psoriasis, hereditary palmoplantar keratoderma, acquired palmoplantar keratoderma, hydradenitis suppurativa, ichthyosis vulgaris, hereditary ichthyosis, acquired ichthyosis, actinic keratosis, a keratinization skin disorder, Gorlin syndrome, nail psoriasis, flexural/inverse psoriasis, Cutaneous T-cell lymphoma and mucosal and nail lesions, comprising administering a topical composition comprising from about 0.01% to about 10% w/w tapinarof and a carrier suitable for topical administration.

2. The method of claim 1, wherein the topical composition comprises from about 0.01% to about 1% w/w, from about 1% to about 3% w/w, from about 3% to about 5% w/w or from about 5% to about 10% w/w tapinarof and a carrier suitable for topical administration.

3. The method of claim 1, wherein the topical composition further comprises a moisturizer, urea, ammonium lactate or combinations thereof.

4. The method of claim 1, wherein the composition further comprises a penetration enhancer.

5. The composition of claim 4, wherein the penetration enhancer is selected from DMSO, propylene glycol, dimethyl isosorbide, isopropyl myristate and combinations thereof.

6. The composition of claim 1, wherein the composition is formulated as a cream, an ointment, a gel, a lotion, a shampoo, a spray, a patch or a foam.

7. The method of claim 1, wherein the skin disorder is selected from palmoplantar psoriasis, hereditary palmoplantar keratoderma and acquired palmoplantar keratoderma.

8. The method of claim 1, wherein the skin or mucosal disorder is selected from a keratinization skin disorder.

9. The method of claim 1, wherein the method comprises once or twice daily topical application of therapeutically effective amounts of the said composition to the skin portion of the subject affected by the said skin or mucosal disorder until the skin or mucosal disorder is cured, prevented or alleviated or according to doctor's instructions.
